(19) United States
(12) Patent Application Publication
(10) Pub. No.: US 2001/0045527 A1
(43) Pub. Date: Nov. 29, 2001

(54) Electron-beam cured polymer mask for DRIE micro-machining

(76) Inventors: Barry Dean Wissman, Ann Arbor, MI (US); Lee Walter, Plymouth, MN (US); Roger Lee Hipwell JR., Eden Prairie, MN (US); Barbara Ihlow-Mahrer, Crystal, MN (US); Zine-Eddine Boutaghou, Vadnais Heights, MN (US)

Correspondence Address: Clifford Chance Rogers & Wells LLP, 200 Park Avenue, New York, NY, 10166-0153, US

(21) Appl. No.: 09/827,560

(22) Filed: Apr. 5, 2001

Related U.S. Application Data
(60) Provisional application No. 60/194,984, filed on Apr. 5, 2000.

(51) Int. Cl. A61N 5/00; G21G 5/00

(52) U.S. Cl. 250/492.2

(57) ABSTRACT

This invention presents a method and system for etching a silicon substrate. This includes depositing a non-thermally cured photoresist mask on the upper region of a trench in the silicon substrate. A fluorocarbon film is deposited on the silicon substrate, and the silicon substrate is bombarded with ions. As a result, the fluorocarbon film is preferentially removed from the lower region of the trench in the substrate, and the upper region of the trench is substantially protected by the photoresist mask. This invention can include curing the photoresist mask using an electron-beam system.

### CROSS-REFERENCE TO RELATED APPLICATION(S)

This application claims the benefit of the filing date of U.S. provisional application serial No. 60/194,984 entitled “Electron-Beam Cured Polymer Mask for DRIE Micro-Machining,” which was filed on Apr. 5, 2000.

### BACKGROUND

In micro-machining of silicon, structures may be anisotropically etched out of silicon substrates to form defined structures, such as trenches, crests, mechanical beams, electrodes, tongues, and flexible ridges. In micro electro-mechanical system devices (“MEMS”), deep, high aspect-ratio trenches for mechanical structures need to be formed in a silicon substrate. A process known as deep-trench reactive ion etching micro-machining (“DRIE”) can be used to create these trenches. The DRIE process requires both high anisotropy and high etch rate. In an etching process, high anisotropy occurs when the etching process has a directional control, i.e., the etching process is applied in a single direction or side so that the etching does not occur in all directions. High etch rate removal is measured by the amount of silicon that is removed from the substrate over time. For example, a DRIE process using a fluorine-rich plasma can result in a high etch rate. Such a process, however, can have poor anisotropy.

These conflicting requirements can be resolved by using a time-multiplexed technique that cycles between etching and deposition of an etch-inhibiting film. This process can result in high anisotropy without sacrificing etch rate. In addition to a high silicon etch rate and anisotropy, the creation of deep, high aspect-ratio trenches by DRIE requires a mask that has an extremely low etch rate relative to the silicon. The use of a masking material with high selectivity can be critical because the silicon etch depth can be hundreds of microns.

A masking material that is customarily used by the MEMS community is an oxide hard mask. Oxide (deposited either thermally or in a plasma) has a high selectivity in the DRIE process. There are numerous problems, however, associated with oxide masks. Deposition of the oxide can require high temperatures and long process times, and is sometimes incompatible with other materials and process steps. Oxide films can also tend to have high inherent stress levels, which can lead to problems such as de-lamination and wafer bowing. In addition, the oxide mask must be patterned and etched, which can add complexity to the process flow. Furthermore, after etching is complete, it can be difficult to strip the oxide mask, which can further complicate the process.

The traditional masking material for conventional reactive ion etching is polymer photoresist. Photoresist is a photo sensitive polymer substance that can chemically react when exposed to light. It is composed of organic polymers, pigments, and fillers. This blend of materials (mostly organic polymers or monomers) is typically applied onto cured wafers and can be part of an images transfer process. Conventionally, the photoresists are cured and dehydrated via a thermal process (e.g., heating and baking). This process is generally accomplished using a modified hot plate on a resist track or an oven. During these operations, the photoresists can crosslink and form a hard organic layer. The strength and extent of the crosslinking can determine its durability during this process. An example of such a process is plasma etching either used in standard RIE or DRIE.

The main advantage of using photoresist is its simplicity. It is easily deposited and patterned and can be readily stripped after etching using solvents or an oxygen plasma. Unfortunately, its inherent selectivity for the DRIE process can be unacceptably low. This can be improved by thermally curing the resist, as the selectivity typically increases with the cure temperature. This, however, can pose other problems. While thermally curing the photoresist above a certain temperature, the photoresist can begin to reflow, which can lead to pattern distortion and can result in loss of dimensional control. In addition, a surface layer can form during curing that can trap residual solvent in the photoresist. Upon subsequent exposure to temperatures greater than the cure temperature (as can occur during the DRIE process), the residual solvent can volatilize and wrinkle or crack the surface, a phenomenon known as reticulation. Furthermore, thermally cured photoresist can have difficulty in corrosive etch environments due to low bond strengths and a low crosslinking concentration.

This invention addresses some of these problems.

### SUMMARY

The present invention provides a method and system for electron-beam cured polymer films as a masking layer for reactive ion etching deep-trench (“DRIE”) micro-machining.

In one aspect of this invention, a method of etching a silicon substrate is presented. The method includes depositing a non-thermally cured photoresist mask on the upper region of a trench in the silicon substrate. A fluorocarbon film is deposited on the silicon substrate, and the silicon substrate is bombarded with ions. As a result, the fluorocarbon film is preferentially removed from the lower region of the trench in the substrate, and the upper region of the trench is substantially protected by the photoresist mask. This method can include curing the photoresist mask using an electron-beam system. The photoresist mask can be removed after the trench is a desired depth by stripping the photoresist mask using a solvent or an oxygen plasma. The fluorocarbon film can include perfluoromethane, CF₄, perfluoroethane, C₂F₆, perfluoropropane, C₃F₈, and perfluorobutane, C₄F₁₀. The photoresist can include diazonapthoquinone photoresist, PMMA, PGMA, and negative based resists (such as polyimides, polyamides, such as SU8). The method can also include flowing the fluorocarbon in a vacuum to deposit the film on the silicon substrate.

In another aspect of this invention, a system for etching a silicon substrate is presented. The system includes a deposited non-thermally cured photoresist mask on the upper region of a trench in the silicon substrate, and a fluorocarbon film deposited on the silicon substrate. The trench is formed by bombardment of the silicon substrate with ions. The fluorocarbon film is preferentially removed from the lower region of the trench in the substrate, and the upper region of the trench is substantially protected by the photoresist mask.

The details of one or more embodiments of the invention are set forth in the accompanying drawings and the description below. Implementations can provide one or more of the following advantages. The use of electron-beam cured polymer masks for DRIE micro-machining can simplify the process flow, and can allow the use of DRIE in devices that cannot tolerate the high temperatures required for oxide deposition. The use of an electron-beam cured polymer mask can also have the advantages associated with the use of a thermally-cured resist mask, but can avoid the issue of thermally-induced pattern distortion, and the corresponding loss of dimensional control. This is vital for MEMS applications where precise control of mechanical features is necessary. In addition, this process can significantly enhance the plasma-resistance of the polymer, allowing the use of thinner masks and more aggressive etching conditions.

### DESCRIPTION OF DRAWINGS

FIG. 1 is a schematic of polymer deposition.

FIG. 2 is a schematic of silicon etching.

FIG. 3 is a flowchart for using electron-beam cured photoresist films as a masking layer for deep-trench RIE micro-machining.

### DETAILED DESCRIPTION

The present invention provides a method and system for electron-beam cured polymer films as a masking layer for reactive ion etching deep-trench (“DRIE”) micro-machining.

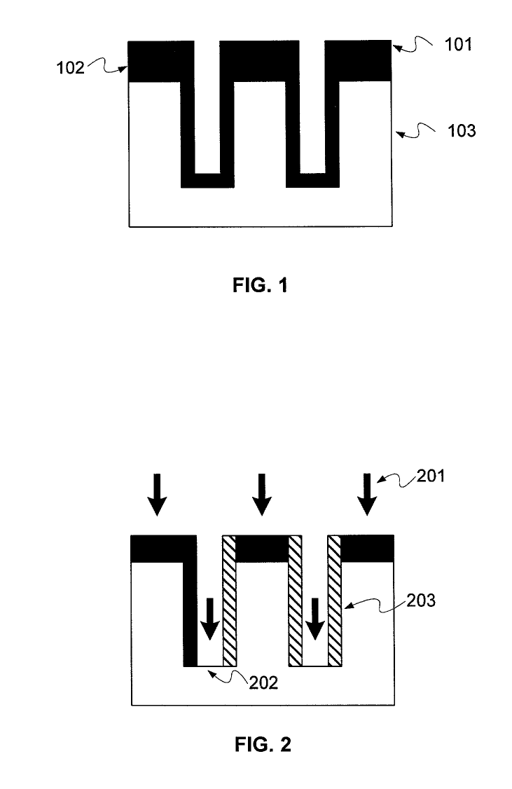

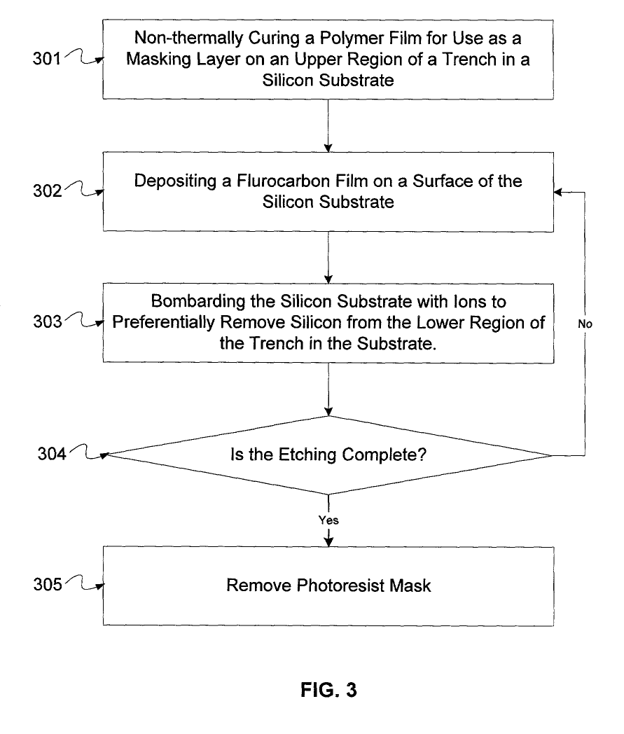

FIG. 3 presents a method for using an electron-beam cured polymer film as a masking layer for DRIE micro-machining. The etching occurs during a time-multiplexed process for DRIE micro-machining. The method cycles between etching and deposition of an etch-inhibiting film. FIGS. 1 and 2 illustrate a schematic representation of the DRIE process. During the deposition cycle, a Teflon-like fluorocarbon film 101 is deposited on the silicon substrate 105. Examples of the fluorocarbon film include, perfluoromethane, CF₄, perfluoroethane, C₂F₆, perfluoropropane, C₃F₈, and perfluorobutane, C₄F₁₀. This can occur by flowing the fluorocarbon in a vacuum to deposit the film on the silicon substrate 105. During the subsequent etching cycle, the film is preferentially removed from the trench bottoms 202 by ion bombardment, allowing the etch to proceed in the vertical direction. At the same time, the fluorocarbon 101 remains on the side walls 203, preventing any etching in the lateral direction. The process can continuously cycle between the polymer deposition and silicon etching with a cycle time of approximately 10-20 seconds. This process can be repeated for hundreds of cycles in order to reach the desired trench depth. In this manner, very high anisotropy can be attained without sacrificing etch rate.

The creation of deep, high aspect-ratio trenches by DRIE requires a mask 102 that has an extremely low etch rate relative to silicon. The mask protects the upper area of the trench from etching. The low etch rate is known as the selectivity and is expressed as the ratio of the silicon etch rate to that of the masking material. The final trench dimensions and profile can depend critically on the mask openings, so any break-down of the mask during etching can lead to a loss of control. The use of masking material with high selectivity can be critical because the silicon etch depth can be hundreds of microns.

For this process, a cured photoresist polymer is used as the masking material. To improve the behavior of this photoresist, stronger bond strengths and higher crosslinking concentrations are required. To achieve this, an enhancement of the photoresist's plasma resistance is required. To enhance the plasma resistance, a beam of electrons is used to provide a non-thermal, low temperature cure. This non-thermally cured photoresist can then be used as a masking material 102 for the DRIE process. Examples of photoresist polymers include diazonapthoquinone photoresist, PMMA, PGMA, and negative based resists (such as polyimides, polyamides, such as SU8).

When curing the photoresist with the use of an electron beam, a higher degree of crosslinking can occur along the track of the electron as it penetrates through the organic media of the photoresist. Additionally, the new bonds formed on recombination of the initial broken fragments are energetically stronger and the distribution through the organic media of the photoresist can be uniform.

The basis of this technique is that, rather than relying on thermal activation, the reactions in the polymer can be stimulated by the kinetic energy of the electrons. The interaction between the polymer and the electrons can create radicals that can then cross-link, which effectively increases the molecular weight of the material. This can be achieved by flood-exposing the substrate to a mono-energetic electron beam. The electron energy (and hence penetration range) can be matched to the resist film's thickness.

The total dose applied can be optimized according to the desired resist properties, and can be uniformly distributed throughout the depth of the film by varying the electron energy during the curing process.

For example, using a typical diazonapthoquinone photoresist cured to an electron dose of 12,0000 μColumbs/cm², a selectivity to silicon of approximately 150 in a DRIE process can be achieved. This selectivity is roughly twice that obtained by thermally curing the same resist at 120° C., and is comparable to that of an oxide-hard mask. Thus, for many MEMS applications, the use of an oxide hard-mask can be avoided. This simplifies the process flow, and allows the use of DRIE in devices that cannot tolerate the high temperatures required for oxide deposition.

After etching is complete, the polymer photoresist can be stripped using solvents or an oxygen plasma 305.

Although the present invention has been described with references to preferred embodiments, workers skilled in the art will recognize that changes may be made in form and detail without departing from the spirit and scope of the invention.

What is claimed is:

1. A method of etching a silicon substrate, the method comprising:
depositing a non-thermally cured photoresist mask on the upper region of a trench in the silicon substrate;
depositing a fluorocarbon film on the silicon substrate; and
bombarding the silicon substrate with ions, wherein the fluorocarbon film is preferentially removed from the lower region of the trench in the substrate, and the upper region of the trench is substantially protected by the photoresist mask.

2. The method of claim 1, additionally comprising:
curing the photoresist mask using an electron-beam system.

3. The method of claim 1, additionally comprising
removing the photoresist mask after the trench is a desired depth.

4. The method of claim 3, wherein the removing the polymer mask comprises stripping the photoresist mask using a solvent.

5. The method of claim 3, wherein removing the polymer mask comprises stripping the photoresist mask using an oxygen plasma.

6. The method of claim 1 wherein the fluorocarbon film comprises a film selected from the group consisting of perfluoromethane, CF₄, perfluoroethane, C₂F₆, perfluoropropane, C₃F₈, and perfluorobutane, C₄F₁₀.

7. The method of claim 1 wherein the photoresist comprises a photoresist selected from the group consisting of diazonapthoquinone photoresist, PMMA, PGMA, and negative based resists.

8. The method of claim 1 wherein the depositing the fluorocarbon film additionally comprises flowing the fluorocarbon in a vacuum to deposit the film on the silicon substrate.

9. A system for etching a silicon substrate comprising:
a deposited non-thermally cured photoresist mask on the upper region of a trench in the silicon substrate; and
a fluorocarbon film deposited on the silicon substrate;
wherein the trench is formed by bombardment of the silicon substrate with ions, the fluorocarbon film is preferentially removed from the lower region of the trench in the substrate, and the upper region of the trench is substantially protected by the photoresist mask.

10. The system of claim 9 wherein the photoresist mask is cured using an electron-beam system.

11. The system of claim 9 wherein the flurocarbon film comprises a film selected from the group consisting of perfluoromethane, CF₄, perfluoroethane, C₂F₆, perfluoropropane, C₃F₈, and perfluorobutane, C₄F₁₀.

12. The system of claim 9 wherein the photoresist comprises a photoresist selected from the group consisting of diazonapthoquinone photoresist, PMMA, PGMA, and negative based resists.

13. A method of etching a silicon substrate, the method comprising:
depositing a fluorocarbon film on the silicon substrate; and
mask means for substantially protecting an upper region of a trench in the substrate from bombardment with ions to form a trench in the silicon substrate.
